(19) United States
(12) Patent Application Publication
(10) Pub. No.: US 2022/0251616 A1
(43) Pub. Date: Aug. 11, 2022

(54) ENDOGLUCANASE AND USE THEREOF

(71) Applicants: OZEKI CORPORATION, Nishinomiya-shi, Hyogo (JP); NATIONAL INSTITUTE OF ADVANCED INDUSTRIAL SCIENCE AND TECHINOLOGY, Chiyoda-ku, Tokyo (JP)

(72) Inventors: Hironori SEMBA, Nishinomiya-shi, Hyogo (JP); Hirokazu TSUBOI, Nishinomiya-shi, Hyogo (JP); Takayuki BOGAKI, Nishinomiya-shi, Hyogo (JP); Akio KODA, Nishinomiya-shi, Hyogo (JP); Hiroyuki YAMADA, Nishinomiya-shi, Hyogo (JP); Kazuhiko ISHIKAWA, Ikeda-shi, Osaka (JP)

(21) Appl. No.: 17/628,898

(22) PCT Filed: Jul. 20, 2020
(86) PCT No.: PCT/JP2020/028051

§ 371 Date: Jan. 20, 2022

(30) Foreign Application Priority Data
Jul. 23, 2019 (JP) 2019-135103

(51) Int. Cl. C12P 19/14; C12Q 1/686

(52) U.S. Cl. CPC C12P 19/14; C12Q 1/686

(57) ABSTRACT

The present invention provides an endoglucanase with excellent heat resistance.

An endoglucanase having characteristics (A) to (C) below:
(A) a polypeptide having an amino acid sequence that is at least 80% identical to the amino acid sequence of SEQ ID NO: 1;
(B) the molecular weight of a polypeptide moiety being about 31 kDa; and
(C) containing a sugar chain, and having a molecular weight measured by SDS-PAGE being about 39 kDa or more.

### TECHNICAL FIELD

The present invention relates to technology involving endoglucanases.

### BACKGROUND ART

Although various techniques for saccharifying cellulose are available, the enzymatic saccharification technique, which requires less energy but produces a high yield of sugar, has been in the mainstream of development. Cellulase, which is a cellulose-degrading enzyme, is broadly divided into cellobiohydrolases, which act on the crystalline regions of cellulose, and endoglucanases, which act inside the cellulose molecular chain to reduce the molecular weight. β-glucosidase acts on a hydrosoluble oligosaccharide or cellobiose to catalyze the hydrolysis of their β-glycosidic bonds.

Endoglucanase (endo-β-1,4-glucanase (EC3.2.1.4)) is an effective enzyme for hydrolytic treatment of cellulose because it hydrolyzes β-1,4-glycosidic bonds between D-glucose, which is a constituent of cellulose. Endoglucanase catalyzes a reaction of endohydrolysis of β-1,4-bonds in not only cellulose, but also cellulose derivatives such as carboxymethylcellulose and hydroxyethylcellulose, lignin, mixed β-1,3-glucans such as cereal β-D-glucans, xyloglucans, and other plant materials containing cellulose moieties.

### CITATION LIST

### Patent Literature

PTL 1: U.S. Pat. No. 4,228,073

### Non-Patent Literature

NPL 1: Kishishita et al., J Ind Microbiol Biotechnol, 2015 42: 137-141

### SUMMARY OF INVENTION

### Technical Problem

Treatment of plant samples or fiber products using an endoglucanase at high temperature attains higher hydrolysis efficiency. If the endoglucanase is not inactivated under high temperature, not only can cellulose be hydrolyzed under high temperature, but also foreign substances such as other enzymes can be inactivated and modified by high-temperature conditions so that an endoglucanase itself that is capable of obtaining a target product at high purity can be efficiently purified. Further, such a heat-resistant endoglucanase can be more efficiently collected and recycled after use. Accordingly, one object of the present invention is to provide an endoglucanase with high heat resistance.

### Solution to Problem

The following describes typical embodiments of the invention.

### Item 1.

An endoglucanase having characteristics (A) to (C) below:

(A) a polypeptide having an amino acid sequence that is at least 80% identical to the amino acid sequence of SEQ ID NO: 1;

(B) the molecular weight of a polypeptide moiety being about 31 kDa; and

(C) containing a sugar chain, and having a molecular weight measured by SDS-PAGE of about 39 kDa or more.

### Item 2.

The endoglucanase according to Item 1, further having the following characteristic (D):

(D) the residual endoglucanase activity after heat treatment at 100° C. for 60 minutes being 30% or more.

### Item 3.

The endoglucanase according to Item 1 or 2, wherein the 104^{th}, 175^{th}, 180^{th}, 221^{st}, 227^{th}, and 258^{th} asparagine residues of the amino acid sequence of SEQ ID No: 1 are conserved.

### Item 4.

A method for producing the endoglucanase according to any one of Items 1 to 3, comprising expressing a DNA encoding the endoglucanase according to any one of Items 1 to 3 using genus Aspergillus as a host.

### Item 5.

A method for producing a reducing sugar, comprising reacting the endoglucanase according to any one of Items 1 to 3 with a sample containing cellulose at a temperature of 70° C. or more.

### Item 6.

A method for isolating the endoglucanase according to any one of Items 1 to 3, comprising exposing the endoglucanase according to any one of Items 1 to 3 to a temperature of 80° C. or more, and subjecting the endoglucanase that has been exposed to a temperature of 80° C. or more to centrifugation.

### Advantageous Effects of Invention

The present invention provides an endoglucanase with high heat resistance. In one preferable embodiment, a means for efficiently producing a reducing sugar from cellulose is provided. In one preferable embodiment, a means for efficiently purifying an endoglucanase is provided.

### BRIEF DESCRIPTION OF DRAWINGS

FIG. 1 shows the results of the molecular weight of endoglucanase measured by SDS-PAGE. The left shows the results of CBB staining, and the right shows the results of PAS staining. Lane M represents a molecular weight marker, 1 represents EGPf expressed in Aspergillus niger, 2 represents EGPf expressed in Aspergillus luchuensis, 3 represents a culture supernatant of Aspergillus niger NS48, 4 represents a culture supernatant of Aspergillus luchuensis NS41, and 5 represents EGPf expressed in E. coli. Each of the portions indicated by the arrow represents the band of EGPf.

FIG. 2 shows the results of effects of a sugar chain on the thermal stability of endoglucanase. Lane M represents the molecular weight marker, 1 represents EGPf without heating, 2 represents EGPf heated at 100° C. for 1 hour, 3 represents EGPf heated at 100° C. for 5 hours, 4 represents EGPf heated at 100° C. for 6 hours, 5 represents EGPf heated at 100° C. for 7 hours, and 6 represents EGPf heated at 100° C. for 8 hours.

### DESCRIPTION OF EMBODIMENTS

### 1. Endoglucanase

The endoglucanase preferably has the following characteristics (A) to (C):

(A) a polypeptide having an amino acid sequence that is at least 80% identical to the amino acid sequence of SEQ ID NO: 1;

(B) the molecular weight of a polypeptide moiety being about 31 kDa; and

(C) containing a sugar chain, and having a molecular weight measured by SDS-PAGE of about 39 kDa or more.

The amino acid sequence represented by SEQ ID NO: 1 is an amino acid sequence (excluding a signal peptide) constituting a wild-type endoglucanase from hyperthermophilic archaeon Pyrococcus furiosus. The identity of the amino acid sequence of the polypeptide of the endoglucanase with the amino acid sequence of SEQ ID NO: 1 is preferably 80% or more, 85% or more, 90% or more, 91% or more, 92% or more, 93% or more, 94% or more, 95% or more, 96% or more, 97% or more, 98% or more, or 99% or more.

The amino acid sequence identity can be determined by using a commercially available analytical tool or an analytical tool available through telecommunication lines (Internet). For example, the amino acid sequence identity can be determined by pairwise alignment using ClustalW Ver. 2.1 (http://clustalw.ddbj.nig.ac.jp/index.php?lang=ja) with default parameters (default setting). Alternatively, the amino acid sequence identity can be determined by using the Basic Local Alignment Search Tool (BLAST; http://www.ncbi.nlm.nih.gov/BLAST/) available from the National Center for Biotechnology Information (NCBI) with default parameters.

The molecular weight of the polypeptide moiety in the endoglucanase is preferably about 31 kDa. The molecular weight calculated from the molecular weight of the constituent amino acid residues of the polypeptide comprising the amino acid sequence of SEQ ID No: 1 is 30522 Da. In one embodiment, about 31 kDa means that the molecular weight calculated from the molecular weight of amine residues constituting a polypeptide is in the range of 30 kDa to 32 kDa. In another embodiment, about 31 kDa means that the molecular weight of the polypeptide moiety alone measured by SDS-PAGE is about 31 kDa.

The endoglucanase preferably contains a sugar chain from the viewpoint of attaining excellent heat resistance. The sugar chain is considered to inhibit a change in steric structure caused by heat and aggregation of the altered polypeptides. Although the amount of the sugar chain in the endoglucanase is not particularly limited, the molecular weight of the sugar-chain-containing endoglucanase measured by SDS-PAGE is preferably about 35 kDa or more, about 36 kDa or more, about 37 kDa or more, about 38 kDa or more, about 39 kDa or more, or about 40 kDa or more. The upper limit of the molecular weight of the sugar-chain-containing endoglucanase measured by SDS-PAGE is not particularly limited; however, it is about 300 kDa, about 200 kDa, about 150 kDa, about 100 kDa, about 90 kDa, about 80 kDa or less, about 70 kDa or less, or about 60 kDa or less. As explained above, the molecular weight of the polypeptide moiety in the endoglucanase is preferably about 31 kDa. Accordingly, the molecular weight of the sugar chain in the endoglucanase is preferably about 4 kDa or more, 5 kDa or more, 6 kDa or more, 7 kDa or more, 8 kDa or more, or 9 kDa or more.

The endoglucanase preferably has a residual endoglucanase activity after heat treatment at 100° C. for 60 minutes of 30% or more. The residual activity rate (%) is measured by comparing the activity of endoglucanase before and after the heat treatment (activity after heat treatment/activity before heat treatment×100). In one embodiment, the residual activity rate is preferably 40% or more, 50% or more, 55% or more, 60% or more, 65% or more, or 70% or more. The heat treatment can be performed by adding the endoglucanase in an amount of 1 U/ml to a sodium phosphate buffer (pH of 7.0) with a final concentration of 200 mM, dissolving or suspending the mixture, setting the temperature to a predetermined temperature in a thermostatic bath, and keeping the temperature for a predetermined time (e.g., 60 minutes).

The endoglucanase activity can be measured by any technique; however, in this specification, it is measured by the Nelson-Somogyi method unless otherwise specified. Specifically, 200 μl of a 50 mM sodium acetate buffer (pH of 5.0) containing a carboxymethylcellulose sodium salt with a final concentration of 1 wt % as a substrate is prepared, a specific amount of endoglucanase is added to the buffer to start the reaction, and the amount of reducing sugar produced at 70° C. for 10 minutes is quantified. By defining the amount of enzyme that releases a reducing sugar in an amount equivalent to 1 μmol of glucose per minute as 1 U, endoglucanase activity per unit weight can be measured.

The endoglucanase may have any mutations in the amino acid sequence of SEQ ID No: 1 as long as it has the desired characteristics as described above. In one embodiment, the endoglucanase preferably conserves at least one of the 104^{th}, 175^{th}, 180^{th}, 221^{st}, 227^{th}, and 258^{th} asparagine residues in the amino acid sequence of SEQ ID No: 1 from the viewpoint of having the above sugar chain. In one embodiment, the endoglucanase preferably conserves at least two, three, four, five, or all of the asparagine residues. In one preferred embodiment, the endoglucanase preferably has all of the asparagine residues. The endoglucanase is considered to have a sugar chain (N-type sugar chain) by bonding the sugar chain to these asparagine residues.

From the viewpoint of maintaining the endoglucanase activity and/or the higher order structure of the protein, the endoglucanase preferably has at least one of the amino acid residues, i.e., Trp at position 35, Trp at position 72, Glu at position 129, Glu at position 148, Tyr at position 194, and Glu at position 241 of the amino acid sequence of SEQ ID NO: 1. Trp at position 35, Trp at position 72, Glu at position 129, and Tyr at position 194 of the amino acid sequence of SEQ ID NO: 1 are considered to involve the active center, and Glu at position 148 and Glu at position 241 are considered to involve substrate binding. In one embodiment, the endoglucanase preferably has at least two, three, four, five, or all of the amino acid residues. In one preferred embodiment, the endoglucanase preferably has all of the amino acid residues described above.

Any technique for adding a mutation to the polypeptide can be used, and the method known in the art can be suitably selected. Examples include a restriction enzyme treatment, treatment using an exonuclease, DNA ligase, etc., site-directed mutagenesis, or random mutagenesis.

The endoglucanase described above can be produced by a genetic engineering technique using DNA as described below. The endoglucanase can also be produced using a general protein chemical synthesis method (e.g., liquid-phase and solid-phase methods) based on information on the amino acid sequence represented by SEQ ID NO: 1.

### 2. DNA Encoding Endoglucanase

The base sequence of a DNA encoding the endoglucanase is not particularly limited. In one embodiment, the DNA preferably has a base sequence with the specific degree of identity with the base sequence of SEQ ID NO: 2. The specific degree of identity refers to, for example, 60% or more, 70% or more, 80% or more, 90% or more, 95% or more, 96% or more, 97% or more, 98% or more, or 99% or more. SEQ ID NO: 2 is the base sequence encoding the amino acid sequence of SEQ ID NO: 1.

The identity of the base sequence can be determined by using a commercially available analytical tool or an analytical tool available through telecommunication lines (Internet). For example, software such as FASTA, BLAST, PSI-BLAST, or SEARCH can be used to determine the identity. The major initial conditions typically applied to a BLAST search are specifically as follows. In Advanced BLAST 2.1, a blastn program is used, and the parameters are set to default values to perform a search, thus calculating the identity (%) of a nucleotide sequence.

In one embodiment, the DNA is preferably present in an isolated state. As used herein, “DNA in an isolated state” means that the DNA is separated from components such as other nucleic acids and proteins that naturally accompany it. However, the DNA may contain a portion of other nucleic acid components, such as nucleic acid sequences that naturally flank the DNA sequence (e.g., the promoter region sequence and terminator sequence). DNAs prepared by a genetic engineering technique, such as cDNA molecules, are, when in an isolated state, preferably substantially free of other components such as cell components and culture media. Likewise, in DNAs prepared by a chemical synthesis, “DNA in an isolated state” preferably means that the DNA is substantially free of precursors (starting materials) such as dNTP, as well as chemical substances, etc., used in the synthetic process.

The DNA can easily be obtained on the basis of the base sequence of SEQ ID NO: 2 by using a chemical DNA synthesis method (e.g., phosphoramidite method) or a genetic engineering technique.

### 3. Vector

The vector preferably includes the DNA in an expressible manner. The type of the vector is suitably selected according to the type of the host cell. Examples of vectors include plasmid vectors, cosmid vectors, phage vectors, and virus vectors (e.g., adenoviral vectors, retroviral vectors, and herpes viral vectors).

Examples of vectors that enable expression in yeast include pBR322, pJDB207, pSH15, pSH19, pYepSec1, pMFa, pYES2, pHIL, pPIC, pA0815, and pPink. Examples of vectors that enable expression in insects include pAc, pVL, and pFastbac. Examples of vectors that enable expression in genus Aspergillus include pSENS2512, pAUR316, pPTR I, and pPTR II.

For a eukaryotic host cell, usable expression vectors include those comprising, at the upstream of the polynucleotide to be expressed, a promoter, an RNA splicing site, a polyadenylation site, a transcription termination sequence, and the like. The expression vectors may further optionally comprise a replication origin, a secretion signal, an enhancer, and/or a selection marker.

### 4. Transformant

The transformant is preferably transformed with the above vector, and those capable of binding the sugar chain to the endoglucanase are preferred. In the transformant, the vector may be present autonomously in the host cell or incorporated into the genome in a homologous or non-homologous recombination manner. From the viewpoint of binding the sugar chain to the endoglucanase, the host is preferably a eukaryotic cell. Examples include yeast such as genus Saccharomyces, genus Pichia, and genus Kluyveromyces, and fungal cells such as genus Aspergillus, genus Penicillium, genus Talaromyces, genus Trichoderma, genus Hypocrea, and genus Acremonium; insect cells, including Drosophila S2, Spodoptera Sf9, and silkworm-culturing cells; and plant cells. It is also possible to produce the endoglucanase in a medium by exploiting the protein secretion capacity of Bacillus subtilis, yeast, fungus, actinomycetes, and the like. In one embodiment, the preferred host is a fungus, more preferably Aspergillus species, and even more preferably Aspergillus niger, Aspergillus luchuensis, Aspergillus oryzae, Aspergillus sojae, Aspergillus nidulans, Aspergillus aculeatus.

To introduce a recombinant expression vector into a host cell, a conventional method can be used. Examples include a variety of methods such as a competent cell method, a protoplast method, an electroporation method, a microinjection method, and a liposome fusion method. However, the method is not limited to these.

The transformant is capable of producing an endoglucanase, and thus can be used for producing the endoglucanase. The transformant itself can also be used for producing reducing sugars, such as glucose, cellobiose, and cello-oligosaccharides from samples containing cellulose.

### 5. Production Method of Endoglucanase Using Transformant

The Endoglucanase can be produced by culturing the transformant and collecting the endoglucanase from the cultured product. The culture can be performed using a passage culture or batch culture with a medium suitable for the host cell. The culture can be performed until a sufficient amount of the endoglucanase is produced, with monitoring the activity of the endoglucanase produced inside and outside of the transformant as a guide.

The culture medium may be suitably selected from conventionally used media according to the type of host cell. The culture can be performed under conditions suitable for growth of the host cell. For the culture of genus Aspergillus, usable examples include nutrient media such as PD medium and DP medium; and minimal media to which a carbon source, a nitrogen source, a vitamin source, and the like are added, such as Czapek-Dox medium.

The culture conditions can be suitably determined according to the type of host cell. The culture is typically performed at 16 to 42° C., preferably 25 to 37° C., for 5 to 168 hours, preferably for 8 to 72 hours. Depending on the host, either shaking culture or static culture can be used, and agitation and/or ventilation may optionally be provided. When an induction promoter is used for gene expression, a promoter-inducing agent may be added to the medium to perform a culture.

Purification or isolation of the endoglucanase from the cultured supernatant can be performed by suitably combining known techniques. Examples of techniques for use include ammonium sulfate precipitation, solvent precipitation (e.g., ethanol), dialysis, ultrafiltration, acid extraction, and a variety of chromatographic approaches (e.g., gel filtration chromatography, anion-exchange or cation-exchange chromatography, phosphocellulose chromatography, hydrophobic interaction chromatography, affinity chromatography, hydroxyapatite chromatography, lectin chromatography, and high-performance liquid chromatography). Examples of carriers used in affinity chromatography include carriers to which an antibody against the endoglucanase is bound and carriers to which a substance with affinity for a peptide tag is bound when the peptide tag is added to the endoglucanase.

When the endoglucanase is accumulated inside the host cells, the transformed cells are disrupted, and the endoglucanase is purified or isolated from the centrifuged supernatant of the disrupted product by the techniques described above. For example, after completion of culture, the cells collected by centrifugation are suspended in a buffer for cell disruption (20 to 100 mM Tris-HCl (pH of 8.0), 5 mM EDTA) and disrupted by ultrasonication. The disruption-treated fluid is centrifuged at 10000 to 15000 rpm for 10 to 15 minutes to thereby obtain a supernatant. The precipitate obtained after centrifugation can optionally be solubilized with guanidinium chloride, urea, or the like, and then further purified.

### 6. Production Method of Reducing Sugars Using Endoglucanase

By the reaction of the endoglucanase with a sample containing cellulose (e.g., a biomass resource), the cellulose is decomposed to produce molasses containing a reducing sugar. Examples of the reducing sugar include glucose, cellobiose, cello-oligosaccharides, and the like. When a biomass resource is used as a sample containing cellulose, it is preferable to use other enzymes such as cellulase in combination with the endoglucanase to produce molasses more efficiently.

The type of the sample containing cellulose is not particularly limited as long as the sample can be decomposed by the endoglucanase. Examples of the sample containing cellulose include bagasse, wood, bran, wheat straw, pasture grasses of Gramineae or Papilionaceae, corncobs, bamboo grass, pulp, rice straw, chaff, wheat bran, soybean meal, soy pulp, coffee grounds, and rice bran.

The temperature at which the endoglucanase is reacted with a sample containing cellulose is preferably 60° C. or more, 70° C. or more, 75° C. or more, 80° C. or more, 85° C. or more, 90° C. or more, 95° C. or more, 98° C. or more, 99° C. or more, or 100° C. or more.

Molasses containing a reducing sugar can be produced from a sample containing cellulose according to a known technique. Biomass resources for use may be either dried materials or wet materials. The materials are preferably milled into particles of 100 to 10000 μm in size beforehand to increase processing efficiency. Milling is performed by using a device such as a ball mill, a vibration mill, a cutter mill, or a hammer mill. The milled biomass resource is immersed in water, steam, or an alkaline solution, and subjected to a high-temperature treatment or a high-temperature high-pressure treatment at 60 to 200° C. to further increase the enzymatic treatment efficiency. For example, alkali treatment can be performed using caustic soda, ammonia, or the like. The biomass sample that has been subjected to such pretreatment is suspended in an aqueous vehicle, and the endoglucanase and cellulase are added thereto, followed by heating with stirring to thereby decompose or saccharize the biomass resource.

When the endoglucanase is reacted with a sample containing cellulose in an aqueous solution, the pH and other conditions in the reaction solution may be within the range in which the endoglucanase is not inactivated.

The molasses containing a reducing sugar may be used unmodified, or may be used as a dry product after removing water. It is also possible to further isomerize or decompose the molasses by a chemical reaction or enzymatic reaction depending on the intended use. The molasses or its fraction can be used, for example, as a starting material for alcohols such as methanol, ethanol, propanol, isopropanol, butanol, and butanediol by a fermentation process.

### 7. Method for Separating Endoglucanase

In the purification of endoglucanase, the endoglucanase-containing sample can be treated at 80° C. or more, thereby inactivating foreign proteins to obtain an endoglucanase with high purity. Further, by treating at 80° C. or more a solution containing foreign substances (e.g., other enzymes or microorganisms) in addition to the endoglucanase, such as a solution obtained after the endoglucanase is reacted with the sample containing cellulose, the foreign enzymes and microorganisms can be inactivated while maintaining the activity of endoglucanase. In one embodiment, the processing temperature can be 80° C. or more, 85° C. or more, 90° C. or more, 95° C. or more, 98° C. or more, or 100° C. or more. The treatment time may be within the range in which the endoglucanase is not inactivated.

The method for separating the endoglucanase can be performed according to a known technique. For example, the endoglucanase and foreign substance can be separated by filtration, centrifugation, microfiltration, rotary vacuum filtration, ultrafiltration, pressurized filtration, cross membrane microfiltration, cross-flow membrane microfiltration, or similar methods.

### EXAMPLES

### 1. Construction of Expression Vector

The endoglucanase gene described in SEQ ID NO: 2 was synthesized and inserted in a plasmid pSENSU (Takaya, T. et al., Appl Microbiol Biotechnol (2011) 90: 1171) that had been previously produced by the inventors. This plasmid contains a secretion signal derived from an α-amylase gene, and can introduce the target gene between the secretion signal and a terminator by PmlI-XbaI treatment. The insertion was conducted as follows.

After the PmlI-XbaI digestion of pSENSU, the pSENSU was subjected to agarose gel electrophoresis to isolate and purify the frayment digested with pSENSU-PmlI-XbaI. Using the synthesized endoglucanase gene described in SEQ ID NO: 2 as a template, insertion frayments were amplified by the PCR method using the primers having base sequences shown in SEQ ID NOs: 3 and 4. The amplified frayments were digested with XbaI and subjected to agarose gel electrophoresis, followed by isolation and purification. The obtained endoglucanase gene was ligated into the PmlI-XbaI site of pSENSU, thus constructing an endoglucanase expression vector pSENSU-EGPf.

### 2. Production of Endoglucanase Using Genus Aspergillus as Host

Using pSENSU-EGPf, Aspergillus niger NS48 (double destruction strain of niaD and sC obtained by mutation treatment) and Aspergillus luchuensis NS41 (double destruction strain of niaD and sC obtained by mutation treatment) strains were transformed by a protoplast-PEG method. Genomic DNAs were extracted from the obtained transformants, thus obtaining transformants with one or more copies of the plasmid introduced into them by a real-time PCR method. These transformants were cultured in dextrin-peptone-yeast extract medium (4 wt % dextrin, 2 wt % polypeptone, 2 wt % yeast extract, 0.5 wt % KH₂PO₄, 0.05 wt % MgSO₄.7H₂O) for 6 days, and the culture supernatants were used as crude enzyme solutions to measure activity. Specifically, the endoglucanase activity was measured as follows. A reaction was started by adding 10 μl of the crude enzyme solution to 200 μl of a 50 mM sodium acetate buffer (pH of 5.0) containing a carboxymethylcellulose sodium salt having a final concentration of 1 wt % as a substrate, and the amount of the reducing sugar generated at 70° C. for 10 minutes was determined by the Nelson-Somogyi method. The amount of the enzyme that releases a reducing sugar in an amount equivalent to 1 μmol of glucose per minute was defined as 1 U, and a strain having an endoglucanase activity of 0.1 U or more per ml of a crude enzyme solution was obtained as an endoglucanase-producing strain with super-heat resistance.

### 3. Production of Endoglucanase Using Escherichia Coli as Host

An expression vector for E. coli containing the endoglucanase gene shown in SEQ ID No: 2 was constructed by a standard method to transform an E. coli BL21 (DE3) strain. The obtained transformant was cultured in an LB medium (1 wt % tryptone, 0.5 wt % yeast extract, 1 wt % sodium chloride, 50 μg/ml carbenicillin), and bacteria collected by centrifugation were lysed with a BugBuster Protein Extraction Reagent (produced by Merck) to extract a crude enzyme solution. The cellulase activity of the extracted crude enzyme solution was measured by the method described in Item 2 above, and a strain having an activity was obtained as a cellulase expression strain with super-heat resistance.

### 4. Estimation of Molecular Weight by SDS-PAGE and Detection of Glycoprotein by PAS Staining

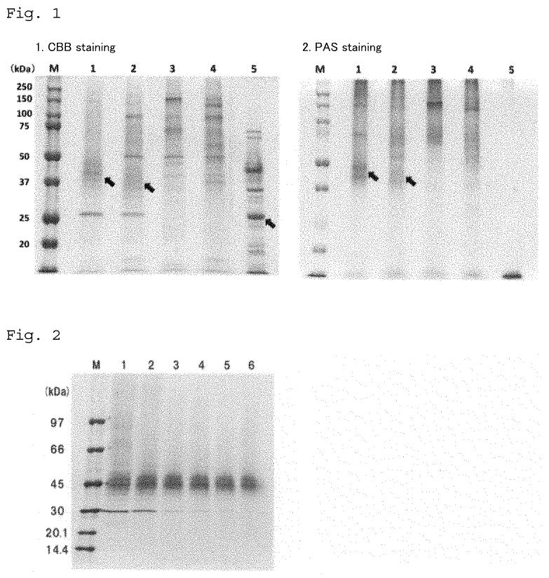

Each of the crude enzyme solutions obtained above was added to a sodium phosphate buffer (pH of 7.0) with a final concentration of 200 mM in such an amount that the endoglucanase concentration was 1 U/ml. The mixture was heated at 80° C. for 30 minutes, and centrifuged at 13,000 rpm for 5 minutes, and the supernatant was obtained as a crude purified enzyme solution. The molecular weight of the super-heat-resistant endoglucanase contained in the crude purified enzyme solution was measured by SDS-PAGE using a molecular weight marker. Consequently, as shown in FIG. 1 on the left, the molecular weight of the endoglucanase produced using Aspergillus niger was 48,500±6,500, the molecular weight of the endoglucanase produced using Aspergillus luchuensis was 44,500±4,500, and the molecular weight of the endoglucanase produced using E. coli was 31,000. Each of the culture supernatants was subjected to SDS-PAGE to detect glycoproteins by PAS staining. As shown in FIG. 1 on the right, the endoglucanases expressed in Aspergillus niger and Aspergillus luchuensis were detected as glycoproteins.

### 5. Evaluation of Thermal Stability

The crude purified enzyme solutions were heated in a heat block at 100° C. for 60 minutes and centrifuged at 13,000 rpm for 5 minutes. Thereafter, the cellulase activity of the supernatant was measured by the above method. As a control, the activity of a sample without heating was measured in the same manner, and the residual activity after heating was calculated as a relative value. The experiment was conducted in triplicate, and the mean value and standard error were calculated. Consequently, as shown in Table 1, the activity of endoglucanase expressed in E. coli was decreased to 13.3% by heating at 100° C. for 60 minutes while the endoglucanase expressed in Aspergillus niger or Aspergillus luchuensis maintained 50% or more of activity even after heating.

TABLE 1
Comparison with heat stability by host

| | Aspergillus | Aspergillus | |
|---|---|---|---|
| | niger | luchuensis | E. coli |
| Residual activity (%) | 72.1 | 62.9 | 13.3 |
| Standard error | 0.59 | 5.20 | 0.44 |

### 6. Evaluation of Effect of Sugar Chain on Thermal Stability

The crude purified enzyme solutions obtained using Aspergillus niger as a host were heated in a heat block at 100° C. for 1 to 8 hours, and centrifuged at 13,000 rpm for 5 minutes, and then the supernatants were subjected to SDS-PAGE. Consequently, as shown in FIG. 2, only bands at around 30 kDa that did not contain a sugar chain disappeared by heating for a long time, while the sugar-chain-containing endoglucanases were unaffected even by heating for a long time.

These results indicate that an endoglucanase containing a specific amount of a sugar chain has significantly higher thermal stability than an endoglucanase that substantially does not contain a sugar chain.

1. An endoglucanase having characteristics (A) to (C) below:
(A) a polypeptide having an amino acid sequence that is at least 80% identical to the amino acid sequence of SEQ ID NO: 1;
(B the molecular weight of a polypeptide moiety being about 31 kDa; and
(C) containing a sugar chain, and having a molecular weight measured by SDS-PAGE of about 39 kDa or more.

2. The endoglucanase according to claim 1, further having the following characteristic (D):
(D) the residual endoglucanase activity after heat treatment at 100° C. for 60 minutes being 30% or more.

3. The endoglucanase according to claim 1, wherein the 104^{th}, 175^{th}, 180^{th}, 221^{st}, 227^{th}, and 258^{th} asparagine residues of the amino acid sequence of SEQ ID NO: 1 are conserved.

4. A method for producing the endoglucanase according to claim 1, comprising expressing a DNA encoding the endoglucanase using genus Aspergillus as a host.

5. A method for producing a reducing sugar, comprising reacting the endoglucanase according to claim 1 with a sample containing cellulose at a temperature of 70° C. or more.

6. A method for isolating the endoglucanase according to claim 1, comprising exposing the endoglucanase to a temperature of 80° C. or more, and subjecting the endoglucanase that has been exposed to a temperature of 80° C. or more to centrifugation.

7. The endoglucanase according to claim 2, wherein the 104^{th}, 175^{th}, 180^{th}, 221^{st}, 227^{th}, and 258^{th} asparagine residues of the amino acid sequence of SEQ ID NO: 1 are conserved.

8. A method for producing the endoglucanase according to claim 7, comprising expressing a DNA encoding the endoglucanase using genus Aspergillus as a host.

9. A method for producing a reducing sugar, comprising reacting the endoglucanase according to claim 7 with a sample containing cellulose at a temperature of 70° C. or more.

10. A method for isolating the endoglucanase according to claim 7, comprising exposing the endoglucanase to a temperature of 80° C. or more, and subjecting the endoglucanase that has been exposed to a temperature of 80° C. or more to centrifugation.

11. A method for producing the endoglucanase according to claim 3, comprising expressing a DNA encoding the endoglucanase using genus Aspergillus as a host.

12. A method for producing a reducing sugar, comprising reacting the endoglucanase according to claim 3 with a sample containing cellulose at a temperature of 70° C. or more.

13. A method for isolating the endoglucanase according to claim 3, comprising exposing the endoglucanase to a temperature of 80° C. or more, and subjecting the endoglucanase that has been exposed to a temperature of 80° C. or more to centrifugation.


SEQUENCE LISTING (4 sequences)

SEQ ID NO: 1 - protein, 270 aa, Pyrococcus furiosus
KVLKIRYPDDGEWPGAPIDKDGDGNPEFYIEINLWNILNATGFAEMTYNLTSGVLHYVQQLDNIVLRDRSNWVHGYPEIFYGNKPWNANYATDGPIPLPSKVSNLTDFYLTISYKLEPKNGLPINFAIESWLTREAWRTTGINSDEQEVMIWIYYDGLQPAGSKVKEIVVPIIVNGTPVNATFEVWKANIGWEYVAFRIKTPIKEGTVTIPYGAFISVAANISSLPNYTELYLEDVEIGTEFGTPSTTSAHLEWWITNITLTPLDRPLIS

SEQ ID NO: 2 - DNA, 813 bp, Pyrococcus furiosus
aaggttctcaagattagataccctgatgacggtgagtggccaggagctcctattgataaggatggtgatgggaacccagaattctacattgaaataaacctatggaacattcttaatgctactggatttgctgagatgacgtacaatttaaccagcggcgtccttcactacgtccaacaacttgacaacattgtcttgagggatagaagtaattgggtgcatggataccccgaaatattctatggaaacaagccatggaatgcaaactacgcaactgatggcccaataccattacccagtaaagtttcaaacctaacagacttctatctaacaatctcctataaacttgagcccaagaacggcctgccaattaacttcgcaatagaatcctggttaacgagagaagcttggagaacaacaggaattaacagcgatgagcaagaagtaatgatatggatttactatgacggattacaaccggctggctccaaagttaaggagattgtagtcccaataatagttaacggaacaccagtaaatgctacatttgaagtatggaaggcaaacattggttgggagtatgttgcatttagaataaagaccccaatcaaagagggaacagtgacaattccatacggagcatttataagtgttgcagccaacatttcaagcttaccaaattacacagaactttacttagaggacgtggagattggaactgagtttggaacgccaagcactacctccgcccacctagagtggtggatcacaaacataacactaactcctctagatagacctcttatttcctaa

SEQ ID NO: 3 - DNA, 31 bp, Artificial sequence
  Primer
ctgctttggctaaggttctcaagattagata

SEQ ID NO: 4 - DNA, 30 bp, Artificial sequence
  primer
gcattctagattaggaaataagaggtctat